(19) United States
(12) Patent Application Publication
(10) Pub. No.: US 2005/0011517 A1
(43) Pub. Date: Jan. 20, 2005

(54) Protective suit ventilated by self-powered bellows

(76) Inventor: Robert Brace Steinert, North Branch, NJ (US)

Correspondence Address: ROBERT B. STEINERT, 32 VILLAGE WAY, NORTH BRANCH, NJ, 08876, US

(21) Appl. No.: 10/621,565

(22) Filed: Jul. 17, 2003

(51) Int. Cl. A62B 17/00; A61M 15/00; A62B 7/00

(52) U.S. Cl. 128/202.19; 128/202.11

(57) ABSTRACT

Protective suits are used to protect the wearer against hazardous materials such as particulates, liquids, chemicals, pathogens, radiation, electromagnetism, physical trauma, heat and for rain protection. They are used to protect the wearer's surrounding in cleanrooms. Medical protective suits are used to prevent the spread of pathogens from the wearer. Unfortunately, they can cause heat stress by reducing air circulation. This invention discloses a protective suit with bellows and dual one-way valves operated by the normal movement of the wearer to draw air through the suit's interior. Filters can be placed in the suit, or the suit can be made of a filter material to protect the wearer and the surroundings. The disclosed suit is lighter, less expensive to produce and more mobile than prior-art protective suit designs that use powered or externally supplied ventilation and cooling devices.

### CROSS REFERENCE TO RELATED APPLICATION

“Ventilated Breathing Powered Protection Suit” specification submitted by Robert B. Steinert, Customer Number 37498.

### FEDERALLY SPONSORED RESEARCH OR DEVELOPMENT

Not Applicable.

### SEQUENCE LISTING

Not Applicable.

### BACKGROUND OF THE INVENTION

Protective suits are in wide use. They are designed to protect the wearer from Hazardous Materials (pathogens, chemicals, particulates, and radioactive contaminates). They are used to protect clean environments from potentially harmful sources from the wearer, such as particular contaminations in cleanrooms. They are used to protect people and animals from pathogens spread by the wearer such as in the surgical operating rooms of medical facilities and for the protection of laboratory animals, which must be pathogen free. They are used to protect workers from electrical, electromagnetic and radioactive sources. They are used for protection from liquids such as chemicals or rain. They are used for physical trauma protection in applications such as construction work, high-speed motor sports and skiing. They are used for heat protection.

The impermeable or semi-permeable construction of these suits is known to limit or eliminate air circulation on the wearer's body, reducing the required body heat loss and evaporation rates. Exhaustion, discomfort and heat stress can result from wearing such suits. To reduce the heat and moisture buildup, the wearer may open the suit's closures to increase ventilation, circumventing the suit's protection.

To reduce these adverse effects, ventilated protection suits of various designs have been disclosed. They use: an external source of supplied air such as compressed air or compressed bottled air, a powered blower carried by the wearer to supply cooling and/or breathing air, or they supply coolants, either gases or liquids, that are contained in circulation devices in the suit's interior. The previously disclosed suits are expensive to produce, add physical weight to the wearer or function for limited periods. Attached supply lines limit the wearer's movement.

Unlike previous designs, this patent discloses the use of a protective suit fitted with a wearer-powered bellows with dual one-way valves which forces or draws air through the protective suit's interior. The air passing through the suit replaces the cooling and evaporation lost when a non-ventilated protective suit is worm. Filtering elements are placed in the air vents prevent harmful elements from entering or leaving the suit. The suit eliminates the need for the powered ventilation or external supplies of air or coolants required by the previously disclosed suits. It is less expensive to produce, lighter to wear, more comfortable and less restrictive of the wearer's movements than the previously disclosed ventilated or cooled suits. It eliminates the tendency to circumvent the protection of non-ventilated or non-cooled protective suits.

U.S. Pat. No. 3,710,395 (Jan. 16, 1973) and U.S. Pat. No. 5,960,475 (Oct. 5, 1999) disclose air permeable garments that provide for air circulation for cooling and evaporation. Their non-filtering construction renders them unsuitable for protective use.

U.S. Pat. No. 6,442,760 (Sep. 3, 2002) and U.S. Pat. No. 5,704,064 (Jan. 6, 1998) disclose ventilated suits that contain open vents in various locations in the suit to allow excess heat and moisture to escape. These openings prevent their use in most situations requiring protective suits. They do not have a mechanism to draw air through the suit. They provide minimal air circulation through the suit, limiting cooling and evaporation.

U.S. Pat. No. 5,564,124 (Oct. 15, 1996) and U.S. Pat. No. 4,903,694 (Feb. 27, 1990) disclose protective suits that use battery-powered blowers to ventilate the suit's interior. Such devices are expensive to produce, heavy and have a limited battery life.

U.S. Pat. No. 4,831,644 (May 23, 1989) discloses a ventilated protective hood attached to a protective suit that uses one-way valves to ventilate the hood and prevent condensation of the hood's window. It does not provide for ventilation of the protective suit.

U.S. Pat. No. 2,255,751 (Sep. 16, 1941), U.S. Pat. No. 2,573,414 (Oct. 30, 1951), U.S. Pat. No. 2,657,396 (Mar. 9, 1951), U.S. Pat. No. 3,292,179 (Dec. 29, 1966), U.S. Pat. No. 4,146,933 (Apr. 3, 1979), U.S. Pat. No. 4,172,454 (Oct. 30, 1979), U.S. Pat. No. 4,194,247 (Mar. 25, 1980), U.S. Pat. No. 4,286,439 (Sep. 1, 1981), U.S. Pat. No. 4,881,539 (Nov. 21, 1989), U.S. Pat. No. 4,458,680 (Jul. 10, 1984), U.S. Pat. No. 5,027,807 (Jul. 2, 1991), U.S. Pat. No. 5,355,857 (Oct. 18, 1994), U.S. Pat. No. 5,339,806 (Aug. 23, 1994) and U.S. Pat. No. 6,209,144 (Apr. 3, 2001) describe protective suits that use external sources for cooling and moisture evaporation, either stationary or carried by the wearer. Typical coolants are compressed bottled air or supplied air, which may also be used for breathing, or refrigerating fluids or gases circulated through the suit. These inventions require the use of a bulky heavy cooling device, carried by the individual, or the use of an external supply line that limits the mobility of the wearer. Their ventilation and cooling sources are expensive to produce. The present invention uses a lightweight bellows with one-way valves and the wearer's normal movement to draw filtered ambient air through the suit to cool the wearer. It does not require an external source of: power, refrigerants for cooling, or air for breathing, cooling and moisture removal.

### SUMMARY OF INVENTION

This invention discloses a ventilated protective suit composed of a permeable, impermeable or semi-permeable overgarment through which fresh air is drawn, or blown, by at least one bellows powered by the movement of the wearer. The bellows have dual one-way valves: the first of the one-way valves allows air to be blown from the bellows, to the suit's interior, when the wearer's movement compresses the bellows, the second valve allows air to be drawn into the bellows from the ambient when the wearer's movement expands the bellows. The opposite flow is also possible, that is, the first valve draws air from the suit into the bellows when the bellows expands and the second valve discharges air to the ambient when the bellows is collapsed. Vents are situated in the suit to allow air to enter, or leave, the suit's interior depending on the arrangement of the one-way valves. The vents are filtered to prevent harmful elements from passing into or out of the suit. The vents are preferably placed near the body's extremities such as the hands, feet and head to expose the maximum area of the body to the fresh air circulation. The suit is designed to prevent collapse, and the potential to lose air circulation, from the negative pressure created when the valves are arranged to draw air through the suit.

The suit can be constructed of materials designed to protect the wearer from hazards such as particulates, pathogens, chemicals, radiation, electricity, electromagnetism, heat, liquids or physical trauma or to protect the surroundings from hazards such as particulates and pathogens.

The bellows can be placed in any position in the suit that allows normal body movements to compress and expand the bellows. A position in the crook of the arm, opposite the elbow between the upper arm and the forearm, is preferable in most circumstances; folding and extension of the forearm is the most likely body movement, providing constant ventilation for normal work. The bellows can also be placed behind the knee, in the crotch of the upper legs or the stomach area or in any other position that produces body movement.

Particulate filters or chemical absorption elements can be placed in the suit's air vents to protect the wearer from potentially harmful agents such as dust, biological pathogens, radioactive materials or chemicals. They also prevent harmful elements from leaving the suit. Particulate filters or chemical absorption elements can be placed in the inlet or outlet ports of inlet or outlet one-way valves to prevent the passage of potentially harmful agents to the wearer or the surroundings and to prevent failure of the valves by an accumulation of contaminants in the valves.

The suit's body can be constructed of permeable or semi-permeable materials, allowing passage of at least some of the incoming fresh air to pass through the suit's body. In this design, the inlet air vents can be reduced in area or eliminated. The permeable or semi-permeable materials can be materials capable of protecting the wearer from hazards such as particulates, pathogens, chemicals, radiation, electricity, electromagnetism, heat, liquids or physical trauma and to protect the surrounding area from hazards such as particulates and pathogens.

The protective suit can be constructed without filtering elements for applications not requiring filtration, to protect the wearer from such sources as: radiation, electricity, electromagnetism, heat, liquids or physical trauma.

Face shields and eye shields, with viewing windows, can be incorporated into the vented suit. The shield(s) can have air vents to allow air to pass through the shield(s) to prevent condensation from fogging the window(s). The shield(s) are connected to the suit's interior by an extension of the suit's body or by any other well-known means, such as a tube or tubes from the shield(s) to the suit's body, to allow the airflow generated by the bellows to pass through the shield(s). Particulate filter or chemical absorption elements can be placed in the shield(s) vents.

The suit in can be used with any of the well-known protective breathing masks such as those with filter elements for breathing ambient air or those supplied with an external source of air such as compressed bottled air or an air supply line. It can be used with a breathing mask that draws air through the protective suit's body such as described in the co-pending patent specification “Ventilated, Breathing-Powered Protection Suit” submitted by Robert B. Steinert, Customer Number 37498.

The disclosed ventilated suit protects the wearer, or adjacent areas. The suit relieves the discomfort, exhaustion and heat stress associated with non-vented non-air-conditioned protective suits. The suit does not require power, coolant or external air. The suit's design provides for a less expensive, lighter weight, more mobile protective suit than the previously known ventilated or cooled suits. It eliminates the tendency to bypass a non-ventilated suit's protective properties by opening the suit's closures.

### DESCRIPTION OF SEVERAL VIEWS OF THE INVENTION

The drawing illustrates a preferred embodiment of the suit that is the object of the present invention.

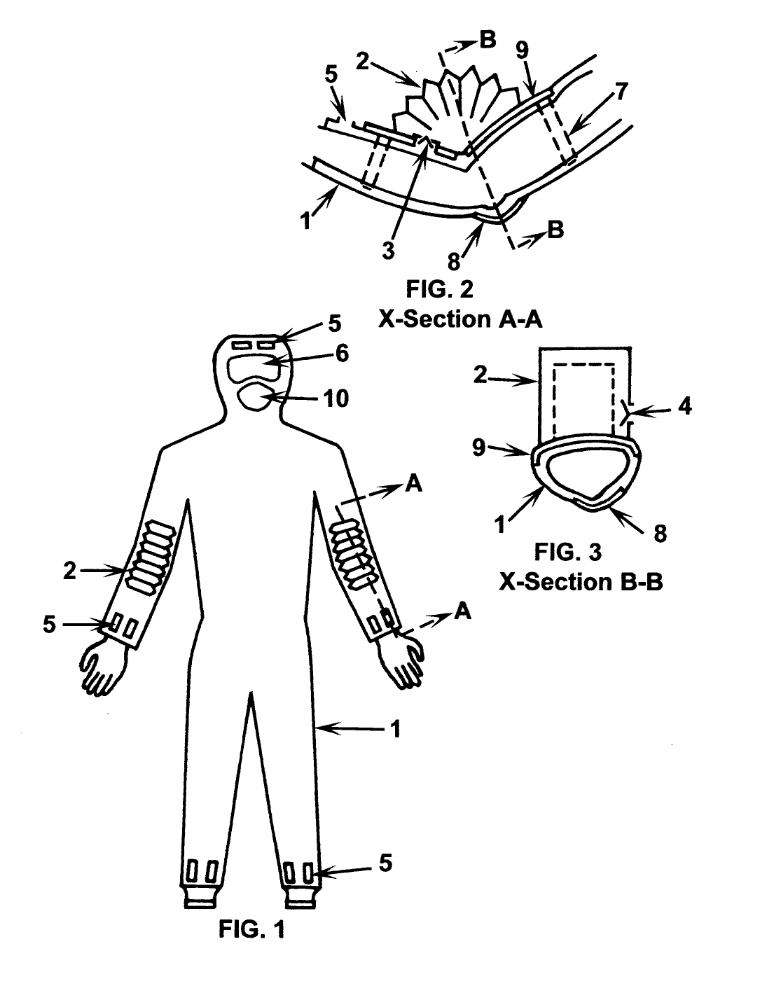

FIG. 1 is a front view of the suit.

FIG. 2 is a cross-sectional view of the suit, parallel to the elbow, through the bellows and the elbow.

FIG. 3 is a cross-sectional view, perpendicular to the arm, through the bellows and elbow.

### DETAILED DESCRIPTION OF THE INVENTION

Item (1) is the suits body, constructed of a permeable, impermeable or semi-permeable material. The suit can cover the head, hands and feet of the wearer as shown.

Item (2) is a bellows that collapses when the wearer's forearm is folded at the elbow and expands when the forearm is extended at the elbow.

Item (3) is a one-way valve (also known as a check valve or automatic valve) that allows the air to flow from the bellows into the suit's interior when the bellows collapses, while preventing reverse flow into the bellows. It can be of any of the well-known designs such as flap valve or ball valve. It can be spring assisted, gravity assisted or mechanically assisted. The valve's ports can be filtered to prevent the escape of potentially harmful material through the valve and to prevent foreign matter from causing valve failure.

Item (4) is a one-way valve (also known as a check valve or automatic valve) which allows air to be drawn from ambient into the bellows when the bellows is expanded, while preventing reverse flow into the suit. It can be of any of the well-known designs such as a flap valve or ball valve. It can be spring assisted, gravity assisted or mechanically assisted. The valve's ports can be filtered to prevent the passage of potentially harmful material from passing through the valve and to prevent foreign matter from causing valve failure.

Items (5) are air ambient air vents that allow air to exit the suit. They are preferably placed near the body's extremities such as the hands, feet and head to allow the maximum exposure of the body to the ventilating air. They can be varied in area to provide for controlled ventilation through various sections of the suit. These vents can be equipped with particulate filters or chemical absorption elements to prevent ingress or egress of harmful materials.

Item (6) is a sight window in the suit's hood.

Items (7) are straps attached to the bellow's base designed to position the bellows to the wearers arm. They can be separate from the suit or part of the suit's body. They can be made of an elastic material or they can be constructed with size adjustments.

Item (8) is an elbow pocket formed in the suit that orients the bellows in the crook of the elbow when the elbow is flexed. It can be made solely from the suit material or it can have addition support material. It can be an integral part of the suit's body or it can be separate from the suit's body, attached with straps to the bellows.

Item (9) is a base plate for the bellows. It is formed partially around the circumference of the forearm and elbow. It positions the bellows when the arm is moved. It is designed to flex in its center in the crook of the arm. It can be designed to extend past the ends of the bellows to provide for additional area to position the bellows and to provide for additional leverage in flexing the bellows. The base of the bellows can be supported by any of the well-known techniques, such as with legs or a three dimensional mesh to allow air to pass under the base of the bellows.

Item (10) is protective breathing mask of any of the well-known designs such as those with: filter elements for breathing ambient air or those supplied with an external source of air such as compressed bottled air or an air supply line. It can be used with a breathing mask that draws air through the protective suit's body such as described in the co-pending patent specification “Ventilated, Breathing-Powered Protection Suit” submitted by Robert B. Steinert, Customer Number 37498.

When the wearer extends their forearms, fresh air is drawn into the bellows from the ambient. When the wearer folds their forearms, the air exits the bellows, enters the suit, and exits the suit through its' vents Forcing fresh air through the suit's interior cools the wearer and evaporates perspiration from the wearer's body.

1) A ventilated wearer-powered protective suit consisting of:
a body constructed of an impermeable, semi-permeable or permeable material capable of protecting the wearer from hazards such as: particulates, pathogens, chemicals, radiation, electricity, electromagnetism, heat, liquids or physical trauma or capable of protecting the surrounding area from hazards such as: particulates and pathogens,
at least one bellows that is operated by the movement of the wearer,
at least one one-way valve, of any of the well-known designs such as a flap valve or ball valve, spring assisted, gravity assisted or mechanically assisted, in the bellows, connected to the suit's body, constructed to allow ambient air to be forced from the bellows through the suit's interior and,
at least one one-way valve, of any of the well-known designs such as a flap valve or ball valve, spring assisted, gravity assisted or mechanically assisted, in the bellows, constructed to allow ambient air to be drawn into the bellows from the ambient,
permanently attached filtering and/or chemical absorbing elements in said valves to prevent harmful elements from passing through the valves or to prevent foreign materials from causing valve failure,
air vents in the suit's body, with permanently attached filtering and/or chemical absorbing elements, to allow ambient air to pass through the suit's interior.

2) A protective suit described in claim (1) that protects any part of the body such as: a suit that fully covers the body or a suit that partially covers the body, such as with uncovered hands, feet or head, or an upper body covering only or a lower body covering only.

3) A protective suit described in claim (1) with a partial face shield or eye shield, a full face shield, or hood covering the head of the wearer, that has:
at least one air vent to allow air to pass through the shield,
at least one air outlet port connected to the suit's interior by an extension of the suit's body, or any other well-known connection technique between the shield and the suit's interior such as a flexible tube, to allow air to pass through the shield and the suit's interior,
a sight window.

4) A protective suit described in claim (1) in which the suit has loose fitting flexible material in the areas between the bellows and the body extremities, such as between an elbow fitted bellows and the wrists, to allow the extremities to move without affecting the bellows position.

5) A protective suit described in claim (1) constructed, at least partially, of a permeable or semi-permeable material that is capable of passing a sufficient a quantity of air through the suit's body to reduce the vent area required in the suit or eliminate the use of air vents in the suit.

6) A protective suit described in claim (1) in which the base of the bellows forms part or the protective suit's body.

7) A protective suit described in claim (1) in which the bellows is constructed separately from the suit's body, with one of the one-way valves connected to the suit's interior by any well-known technique such as an extension of the suit's material or a tube.

8) A ventilated protective suit described in claim (1) in which said bellows are placed in any position on the wearer's body in which movement of the wearer's body can expanded and collapsed the bellows, such as: the crotch of the elbow, the crotch of the knees, the buttocks, the crotch of the upper legs or in front of the hips.

9) A protective suit described in claim (1) in which the bellows is of any of the well-known designs such as: a fan shaped bellows in which the corrugations meet at an apex, an expandable corrugated tube, a rectangular bellows such as used in an accordion or any expandable and collapsible enclosed shape such as an elastic flexible tube that self expands when forces are removed from it, a blatter or a sphere.

10) A protective suit described in claim (1) in which the bellows has a base plate to hold the bellows in place when the wearer moves, with:
the base plate formed to fit the circumference of the wearer's body part supporting the bellows,
the ends of the base plate extended past the ends of the bellows,
a flexible joint in the base plate in the crook of the joint operating the bellows.

11) A protective suit described in claim (1) in which the bellows are held in place by straps, either self-adjusting elastic straps or adjustable length straps, built into the suit's material or separate from the suit's material.

12) A protective suit described in claim (1) in which the bellows is held above the wearers body, to allow air to pass under and around the bellow's base, using any of the well-known techniques such as a three dimensional mesh, a double layered base or supports.

13) A protective suit described in claim (1) in which a blatter, or blatters, with one-way valves, is placed between the upper legs to allow the blatter(s) to expand and collapse when the wearer walks.

14) A protective suit described in claim (1) which has pockets located over the body joints (such as the elbow) opposite the bellows to orient the bellows when the joint is folded.

15) A pocket in claim (14) made solely from the suit's material, or with addition reinforcement material in the pocket, that is either an integral part of the suit's body or separate from the suit's body, attached to the bellows by any of the well-known techniques such as with the use of straps or the suit's material.

16) A ventilated protective suit described in claim (1) in which the bellows has:
at least one one-way valve, connected to the suit, designed to allow air to be drawn from the suit's interior into the bellows and,
at least one one-way valve designed to force air from the bellows to the ambient.

17) A protective suit described in claim (16) that is constructed to allow the passage of air through the suit when it collapses under the negative pressure created when air is drawn through the suit, using any well-known technique such as: stays, reinforcing loops, convolutions, three dimensional lining, such as mesh, batting, netting or tubes that allows air to pass through the suit's interior.

18) A protective suit described in claim (1) which has more than one one-way valve element in the one-way valves, connected in series, to allow for continued operation of the valves if one of the valve elements fails open (fails to close off the reverse flow of air).

19) A suit in claim (1) constructed without filtering elements in the valve ports, to protect the wearer from harmful sources not requiring filtration such as: radiation, electricity, electromagnetism, heat, liquids or physical trauma.

20) A protective suit described in claim (1) that contains replaceable particulate filtration and/or chemical absorption elements in the one-way valves.

21) A protective suit described in claim (1), in which the vents that allow air to pass through the interior of the suit, are areas that are commonly designed to be open, such as:
the end of the legs and arms, the waist of a jacket, under arm vents, back vents and closures such as zippers and buttons.

22) A suit in claim (1) constructed without filtering elements in the air vents, to protect the wearer from harmful sources not requiring filtration such as: radiation, electricity, electromagnetism, heat, liquids or physical trauma.

23) A protective suit described in claim (1) that contains replaceable filtration and/or chemical absorption elements in the air vents.

24) A protective suit described in claim (1) in which the air vents are shielded to prevent entry of liquids such as rain or chemicals.

25) A protective described suit in claim (1) that has:
a protective breathing mask with permanent or replaceable filtering elements or,
a protective breathing mask with an external source of air such as compressed bottled air or an air supplied by an attached line or,
a breathing mask with dual one-way valves that draws air from, or forces air through, the protective suit's body such as described in the co-pending patent specification submitted by Robert B. Steinert, Customer Number 37498.
